(19) United States
(12) Patent Application Publication
(10) Pub. No.: US 2025/0043223 A1
(43) Pub. Date: Feb. 6, 2025

(54) DEVICE FOR PROVIDING AND HANDLING A STERILE LIQUID MEDIUM, METHOD FOR PROVIDING A LIQUID CULTURE MEDIUM AND USE OF A CORRESPONDING DEVICE FOR SAMPLING IN CLEANROOMS OR ISOLATORS

(71) Applicant: PharmaMedia Dr. Mueller GmbH, Leimen (DE)

(72) Inventors: Rolf Mueller, Dossenheim (DE); Heinz Schindler, Walldorf (DE); Ulrich Eikmanns, Bensheim (DE)

(73) Assignee: PharmaMedia Dr. Mueller GmbH, Leimen (DE)

(21) Appl. No.: 18/923,454

(22) Filed: Oct. 22, 2024

Related U.S. Application Data
(63) Continuation of application No. PCT/DE2023/200169, filed on Aug. 17, 2023.

(30) Foreign Application Priority Data
Nov. 22, 2022 (DE) 10 2022 212 454.5

(51) Int. Cl. C12M 1/00; C12M 1/12

(52) U.S. Cl. CPC C12M 29/00; C12M 23/22; C12M 23/32; C12M 37/04

(57) ABSTRACT

The disclosure relates to a device for providing and handling a sterile liquid medium, including a closed ampoule containing the medium and one swab composed of a rod and a swab head, the ampoule and the swab are arranged opposite each other in a housing for storage and handling. The housing is openable in the region of the swab to release the swab head for use and closable after use of the swab head, and the ampoule is openable in the closed housing so that the medium reaches and surrounds the swab head inside the housing. The disclosure also provides a method for providing a liquid culture medium, in particular a liquid nutrient medium for cultivation of microorganisms, preferably pathogens, bacteria, fungi and yeasts, and/or viruses, preferably for use in cleanrooms or isolators. The disclosure lastly relates to the use of a corresponding device in cleanrooms or isolators.

### CROSS-REFERENCE TO RELATED APPLICATIONS

This application is a continuation application of International Patent Application No. PCT/DE2023/200169, filed on Aug. 17, 2023, which claims priority to German Patent Application No. 102022212454.5, filed on Nov. 22, 2022, the entire contents of each of which is incorporated by reference herein in its entirety.

### BACKGROUND

### 1. Field of the Disclosure

The disclosure relates to a device for providing and handling a sterile liquid medium or a sampling solution, in particular for sampling in cleanrooms or isolators. The disclosure further relates to a method for providing a liquid culture medium/sampling solution, in particular a liquid nutrient medium for cultivation or detection of microorganisms, preferably pathogens, bacteria, fungi and yeasts, and/or viruses, preferably for use in cleanrooms, isolators or comparable production and test spaces, referred to hereinafter as cleanroom areas. The disclosure lastly relates to the use of a corresponding device in cleanrooms or isolators.

### 2. Description of the Related Art

Cleanrooms or aseptically operated insulators are well known from practice. For example, sterile production of active ingredients in the pharmaceutical industry or in biotechnology can be carried out there. Cleanrooms or isolators can not only protect the product concerned from environmental influences but may also protect the operator from highly active substances. Therefore, cleanrooms and isolators are widely used in the final stage of production of sterile pharmaceutical substances.

The checking of cleanroom areas (cleanrooms, isolators or comparable production and test spaces) requires that they be constantly tested for the presence of pathogens or bacteria. This is typically done with so-called contact plates in the case of smooth and easy-to-reach surfaces. In the case of harder-to-reach surfaces or irregularly shaped surfaces, this testing is typically done with the aid of sterile swab tubes, which comprise a swab comprising a rod and a cotton wad-like swab head. Such an arrangement is usually referred to as a cleanroom swab. After swabbing, it is necessary to contact the swab head with a nutrient medium/detection medium in order to be able to detect the development/detection of pathogens/bacteria and, optionally, in order to perform a qualitative and quantitative determination of pathogens. The handling of most swabs known from practice is complicated and thus inconvenient. In addition, there is always the risk of contamination due to handling. Typically, the products currently on the market are filled aseptically and subsequently irradiated, and so the possibility of contamination cannot be ruled out, meaning that false-positive results may occur.

### SUMMARY

It is therefore an object of the disclosure to specify a device for biocontamination control that does not have the disadvantages which occur in the prior art. The device is to be simple in design and simple to handle and is to be different from competitor products.

Furthermore, a method for providing a liquid culture medium with use of the device according to the disclosure is to be specified. Likewise, the corresponding use in cleanrooms, isolators or comparable production and test spaces (cleanroom areas) is to be specified.

The above object is achieved by the features of claim 1 with respect to the device according to the disclosure. According thereto, the device comprises at least one closed ampoule containing the liquid medium and one swab composed of a rod and a swab head formed at one end of the rod. The ampoule and the swab are arranged opposite each other in a housing serving for storage and handling, wherein the housing is openable in the region of the swab to release the swab head for use thereof and re-closable after use of the swab head. The ampoule is openable in the closed housing in such a way that the medium reaches and surrounds the swab head inside the housing.

According to the disclosure, there is provided a device in which the swab product serves both for providing and handling the swab and for providing the liquid nutrient medium. Thus, the swab can be closed in the housing after use thereof and the swab head is surrounded—in situ—by a liquid nutrient medium, meaning that there is no need for separate handling of the swab and/or the nutrient medium/detection solution. Thus, the device can also be used for cultivation of bacteria/pathogens.

Specifically, the housing comprises a first housing part containing the ampoule and a second housing part surrounding the swab, the rod of the swab being anchored to or in the first housing part, for example being plugged therein. The two housing parts are arranged axially or coaxially to each other as an elongated sleeve closed at the ends.

The housing parts can be easily plugged together or screwed together or sealingly connected to one another by means of a bayonet connection. What is essential is the sealing connection between the two housing parts, thereby yielding the housing as a whole.

The first housing part comprises a space for accommodation of the ampoule and comprises a coupling region firstly for end-side accommodation of one end of the ampoule, optionally for fixing of the ampoule, and secondly for opposite fixing of the free end of the rod of the swab and for accommodation or attachment of the second housing part. The coupling region forms effectively a spatial separation between the two housing parts, there being a fluid connection for fluid through the coupling region.

The open end of the second housing part is pluggable into the open end of the first housing part or vice versa, preferably up to a mutual stop which limits the distance of plugging in.

Formed between the two housing parts, in particular through the coupling region of the first housing part, is a flow connection through which a fluid can flow or communicate between the housing parts. It is also conceivable that the flow connection extends at least through part of the rod of the swab, and so the fluid can, for example, issue into the interior of the second housing part in the vicinity of the swab head. The flow connection realized is essential.

The ampoule comprising the liquid culture medium is preferably in the form of a glass or plastic ampoule and has at least one predetermined breaking point at one end. The ampoule can also be an ampoule in which each of the two ends has a predetermined breaking point for opening of the ampoule. Opening the ampoule at both ends has the advantage that the liquid culture medium can easily issue from the ampoule without any negative pressure arising in the ampoule preventing the liquid from issuing. It is also conceivable that the ampoule comprises two interior spaces for provision of two different media.

Further advantageously, the first housing part is reduced in diameter and/or elastic in the region of the predetermined breaking point(s) of the ampoule in such a way that the ampoule is openable and the liquid medium thereby releasable by bending and/or compression of the first housing part. It can issue from the ampoule unimpeded, specifically into the first housing part first of all. From there, the fluid passes through the coupling region into the second housing part, where it can contact or surround the swab head.

The first housing part is closed at its free end, i.e., on the side facing away from the coupling region, either reversibly by means of a closure or irreversibly by welding, compression or bonding. What is essential here is that the first housing part is sealingly closable after the ampoule filled with liquid medium has been introduced.

Very particularly advantageously, the first housing part has in the region of its end facing the second housing part a structure formed on the surface, for example a fluting or the like. This facilitates handling in terms of a grip. The provision of a certain surface roughness already suffices for this purpose.

The two housing parts are preferably made by injection molding from plastic in particular. It is very particularly advantageous if the second housing part consists of a see-through plastic, so that the swabs head present in the second housing part and the culture medium can be observed.

As already stated above, the housing parts are sealingly connected to one another or they can be sealingly connected to one another to rule out the possibility of contaminated medium reaching the outside of the housing or, alternatively, of contamination passing from the outside to the inside after sampling.

In line with the foregoing, the medium is a liquid nutrient medium for cultivation of microorganisms, in particular pathogens, bacteria, fungi, yeasts, and/or viruses, depending on the specific application.

Likewise, the medium can be an analysis or buffer solution for any further processing.

It is also conceivable that the medium is mixed with a deinhibitor or neutralizer, for example for neutralization of a disinfectant or the like, which may be present on the surfaces to be swabbed. In the absence of such a neutralizer, the result of culturing would be impaired.

The method according to the disclosure achieves the object mentioned at the start by the features of the additional independent claim 17, according to which the method serves for providing a liquid culture medium, in particular a liquid nutrient medium for cultivation of microorganisms, in particular pathogens, bacteria, fungi and yeasts, and/or detection of viruses or other sample material. Use in cleanrooms or isolators can follow the mere provision, wherein either the culture medium is filled into an ampoule aseptically and/or the ampoule, after it has been filled and closed, is sterilized under the action of heat, preferably steam, and then under the action of radiation, preferably gamma radiation. The method serves for use of the above-discussed device.

The method uses the device according to the disclosure, wherein said device can be completed as follows:

Step 1
1. the ampoule is filled with a culture medium;
2. the filled and closed ampoule is inserted into a first housing part, wherein the insertion-side opening of the first housing part is closed by means of a closure or by welding or by compression;

Step 2
a swab is plugged into a slot of the first housing part opposite the opening,
wherein the first housing part serves as a handle for handling of the swab;

Step 3
a second housing part is marked or labelled;

Step 4
the swab is moistened with a solution; thereafter, the first and second housing parts including the swab are plugged together;

Step 5
the device containing the swab is individually packed or a plurality thereof is packed;

Step 6
2 to 10 individual packs, preferably 5 individual packs, are packed in a first outer pack (can also be more or less individual packs);

Step 7
an irradiation indicator is attached to at least one of the outer packs, preferably to the first outer pack; the attachment of the irradiation indicator is not absolutely necessary.

Step 8
the first outer pack with the individual packs contained therein is placed into a second outer pack or into multiple outer packs;

Step 9
the entire arrangement in the second outer pack is placed into a carton; the carton has been printed on or is printed on or it has been labelled or is labelled;

Step 10
the carton with the outer packs/individual packs placed therein and the devices plus swabs present therein is/are irradiated and/or autoclaved and/or gas-treated and/or sterilized in some other way, at least with a reduction in pathogens.

Furthermore, the object mentioned at the start is achieved by the further additional independent claim 19 with respect to the use according to the disclosure. According thereto, the packed device including the swab present therein is introduced into a cleanroom zone or into an insulator classified according to the guidelines for cleanroom classes as A, B, C or D or ISO, 8, 7, 6 or 5 or better, wherein the swab is removed from the innermost pack and critical zones or zones to be tested are sampled or swabbed using the swab, wherein the swab is then plugged back into the first housing part, wherein the ampoule is broken open at one end or at both ends, so that the solution present therein runs into the second housing part toward the swab head, wherein the liquid either flows past the rod of the swab or else flows through an inner channel in the rod of the swab and wherein the solution is directly incubated, for example in a laboratory, or immediately after use or afterwards the solution is completely or partially removed from the housing and is used for other applications, for example for filtration, for application of molecular biology methods, for identification, etc.

There are, then, various ways of embodying and developing the teaching of the present disclosure in an advantageous manner. To this end, reference can be made to the claims subordinate to claim 1 and to the following elucidation of a preferred exemplary embodiment of the device according to the disclosure by means of the drawing. In connection with the elucidation of the preferred exemplary embodiment of the device according to the disclosure by means of the drawing, preferred embodiments and developments of the teaching will also be elucidated in general. In the drawing

### BRIEF DESCRIPTION OF THE DRAWINGS

FIG. 1 shows a schematic view of one exemplary embodiment of a device according to the disclosure, wherein the device is shown in an external view.

FIG. 2 shows a schematic view of the exemplary embodiment according to FIG. 1 rotated by 90° about the longitudinal axis.

FIG. 3 shows a sectional view of the object from FIGS. 1 and 2, according to which the interior of the two housing parts can be seen.

FIG. 4 shows the object from FIGS. 1 to 3 with the second housing part removed, wherein the swab is in its exposed use position.

FIG. 5 shows the object from FIGS. 1 to 3 with the second housing part inserted, wherein the swab is in its protected provision/transport position.

### DETAILED DESCRIPTION

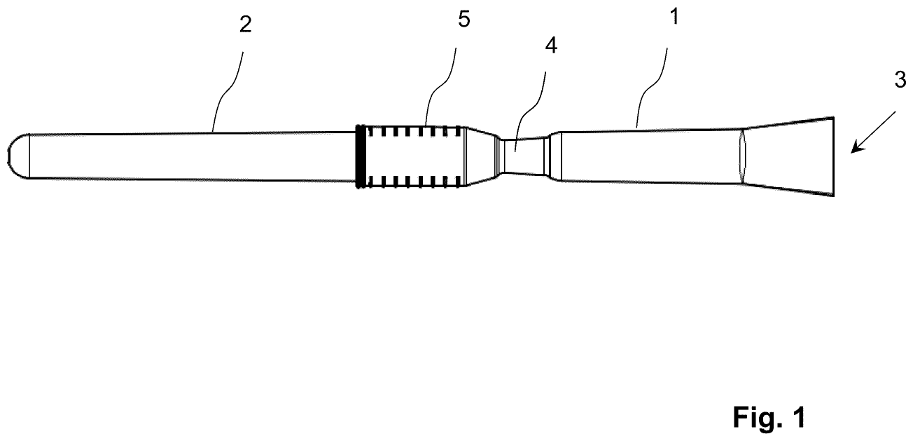

FIG. 1 shows a schematic view of one exemplary embodiment of a device according to the disclosure. The device comprises a first housing part 1 and a second housing part 2, the second housing part 2 being sealingly plugged into the first housing part 1.

The first housing part 1 is closed at its free end, an ampoule having been introduced through an opening 3 prior to closure.

On the opposite side, the first housing part 1 is reduced in cross-section, a coupling region 4 being present therein.

At the free end of the first housing part 1, facing the second housing part 2, the first housing part 1 can be provided with a fluting 5, which fluting serves as a grip.

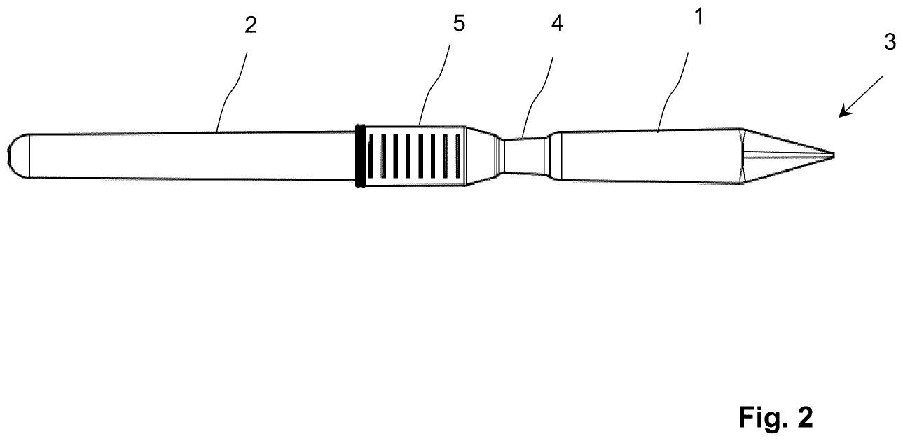

FIG. 2 shows the object from FIG. 1 rotated by 90° about the longitudinal axis, the free end of the first housing part 1 being closed by bonding or by welding. The ampoule, which is not shown, is present inside the first housing part 1. Instead of bonding/welding, a sealing closure can be provided at the free end of the first housing part 1.

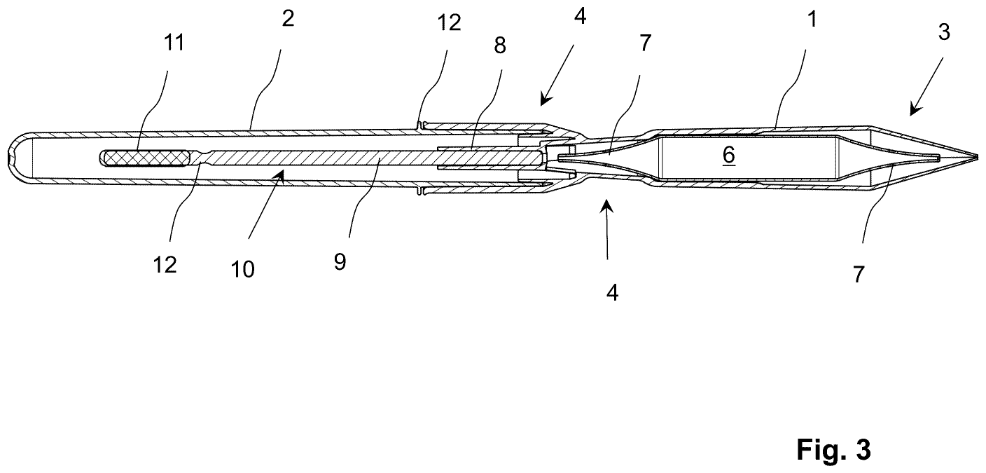

FIG. 3 shows a sectional view of the object from FIGS. 1 and 2. It can be clearly seen here that present in the first housing part 1 is an ampoule 6 which can have, at both ends, a predetermined breaking point 7 in each case for opening of the ampoule 6. The ampoule 6 consists of glass or another suitable material. Accordingly, the ampoule 6 can be opened by breakage of the predetermined breaking point 7.

Furthermore, FIG. 3 clearly shows that the ampoule 6 extends into the coupling region 4. It can be plugged therein or anchored there in some other way by one of the two predetermined breaking points 7.

The coupling region 4 comprises a flow connection between the first housing part 1 and the second housing part 2, and the flow connection can be coaxial in the second housing part 2.

Furthermore, it can be seen from FIG. 3 that provided within the coupling region 4 is a slot 8 into which the rod 9 of the swab 10 is form-fittingly and/or frictionally connected, for example plugged in, bonded or welded. In any case, the swab 10 has a firm hold in the slot 8.

The rod 9 of the swab 10 extends up to the free end, on which a swab head 11 is formed as a cotton wad or the like.

Furthermore, it can be seen in FIG. 3 that the second housing part 2 can be provided with a stop 12, which extends annularly around the second housing part 2. The stop 12 serves as a slide-in limit with respect to the first housing part 1, and so this defines the plugging of the second housing part 2 into the first housing part 1 (or vice versa).

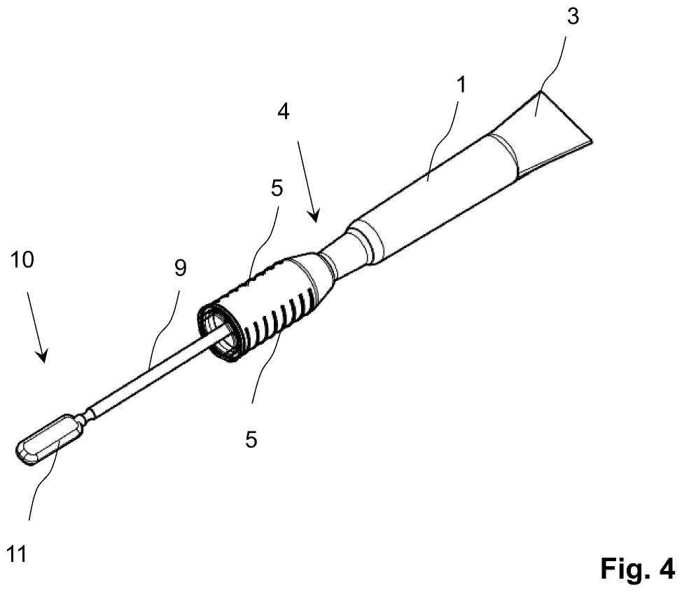

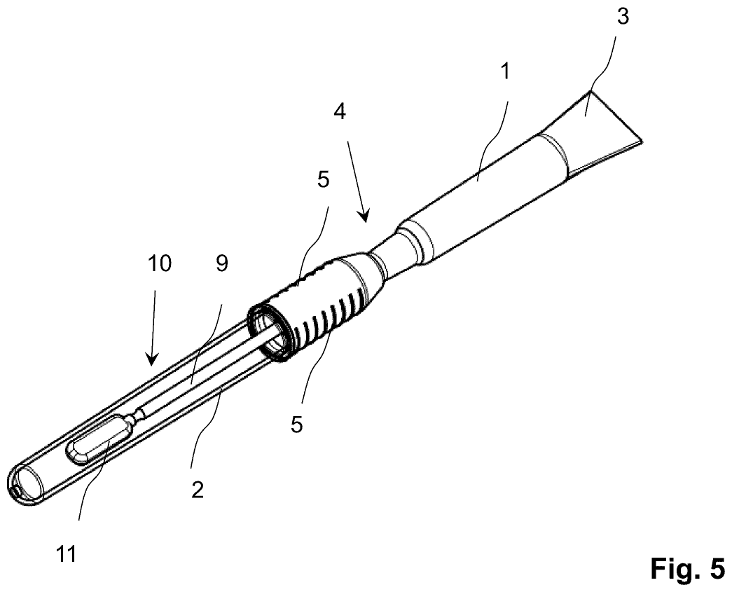

FIG. 4 shows the device according to the disclosure in the use position or use situation, according to which the second housing part 2 is removed. Thus, the swab head 11 is exposed and can be used, held on the first housing part 1. After use or after swabbing of critical surfaces, the device can be closed as per the illustration in FIG. 5, namely by insertion of the second housing part 2 and closure of the device as a result.

As a result of opening of the ampoule not shown in FIG. 5, the liquid medium is released and, in accordance with the positioning of the entire device, reaches the region of the second housing part 2, specifically the swab head 11, and so the swabbed bacteria can develop in the medium, generally a nutrient medium/culture medium.

With respect to further advantageous embodiments of the device according to the disclosure, reference is made to the general part of the description and to the accompanying claims to avoid repetition.

Lastly, it should be expressly pointed out that the above-described exemplary embodiment of the device according to the disclosure merely serves for discussion of the claimed teaching, but does not restrict it to the exemplary embodiment.

### LIST OF REFERENCE SIGNS

1 first housing part
2 second housing part
3 opening, closure
4 coupling region
5 fluting
6 ampoule
7 predetermined breaking point
8 slot
9 rod
10 swab
11 swab head
12 stop

The invention claimed is:

1. A device for providing and handling a sterile liquid medium, comprising:
at least one closed ampoule containing the medium and one swab composed of a rod and a swab head formed at one end of the rod, wherein the ampoule and the swab are arranged opposite each other in a housing for storage and handling, wherein the housing is openable in the region of the swab to release the swab head for use thereof and closable after use of the swab head, wherein the ampoule is openable in the closed housing so that the medium reaches and surrounds the swab head inside the housing, wherein the housing comprises a first housing part containing the ampoule and a second housing part surrounding the swab, wherein the housing parts are arranged axially or coaxially to each other as an elongated sleeve closed at the ends, and wherein the first housing part comprises a space for accommodation of the ampoule and comprises a coupling region firstly for end-side accommodation of one end of the ampoule for fixing of the ampoule, and secondly for opposite fixing of the free end of the rod of the swab and for accommodation or attachment of the second housing part.

2. The device according to claim 1, wherein the housing parts are plugged together or screwed together or sealingly connected by a bayonet connection.

3. The device according to claim 1, wherein the open end of the second housing part is pluggable into the open end of the first housing part or vice versa, up to a stop.

4. The device according to claim 1, wherein between the two housing parts a flow connection is formed through the coupling region of the first housing part, so that a fluid to flow between the housing parts through the flow connection is possible.

5. The device according to claim 1, wherein the ampoule is in the form of a glass or plastic ampoule having at least one predetermined breaking point at one end, or having predetermined breaking points at both ends, for opening of the ampoule, and wherein the first housing part is reduced in diameter and/or elastic in the region of the predetermined breaking point(s) of the ampoule so that the ampoule is openable and the medium thereby releasable by bending and/or compression of the first housing part.

6. The device according to claim 1, wherein the first housing part is closed at its free end on the side facing away from the coupling region, either reversibly by closure or irreversibly by welding, compression or bonding.

7. The device according to claim 1, wherein the first housing part can have in the region of the end facing the second housing part a structure formed on the surface, which facilitates handling.

8. The device according to claim 1, wherein the housing parts are made by injection molding from plastic.

9. The device according to claim 1, wherein the second housing part consists of a see-through plastic.

10. The device according to claim 1, wherein the housing parts are sealingly connected or connectable to one another.

11. The device according to claim 1, wherein the medium is a liquid nutrient medium for cultivation of microorganisms, and/or a medium for sampling and for detection of viruses, or the medium is an analysis or buffer solution for any further processing.

12. The device according to claim 2, wherein the open end of the second housing part is pluggable into the open end of the first housing part or vice versa, up to a stop.

13. The device according to claim 2, wherein between the two housing parts a flow connection is formed through the coupling region of the first housing part, so that a fluid to flow between the housing parts through the flow connection is possible.

14. The device according to claim 11, wherein the medium is mixed with a deinhibitor or neutralizer.

15. A method for providing a liquid culture medium or a sampling medium for the cultivation of microorganisms, wherein the microorganisms are pathogens, bacteria, fungi and yeasts, and/or viruses, for use in cleanrooms or isolators; and
wherein either the culture medium is filled into an ampoule aseptically and/or the ampoule after it has been filled and closed and is sterilized under the action of heat and then under the action of radiation, for use in a device of claim 1.

16. The method according to claim 15, wherein the device is completed according to the following steps, and wherein an order of the following steps 1, 2 and 3 is freely selectable:
Step 1)
1. the ampoule is filled with a culture medium/sampling medium/transport medium; and
2. the filled and closed ampoule is inserted into a first housing part, wherein the insertion-side opening of the first housing part is closed by a closure or by welding or by compression;
Step 2)
a swab is plugged into a slot of the first housing part opposite the opening, wherein the first housing part serves as a handle for handling of the swab;
Step 3)
a second housing part is marked or labelled;
Step 4)
the swab is moistened with a solution; thereafter, the first and second housing parts including the swab are plugged together;
Step 5)
the device containing the swab is individually packed or a plurality thereof is packed;
Step 6)
2 to 10 individual packs are packed in a first outer pack;
Step 7)
an irradiation indicator is attached to the first outer pack;
Step 8)
the first outer pack with the individual packs contained therein is placed into a second outer pack or into multiple outer packs, wherein the outer pack is suitable for cleanrooms/insulators;
Step 9)
the entire arrangement in the second outer pack is placed into a carton; the carton has been printed on or is printed on;
Step 10)
the carton with the outer packs/individual packs placed therein and the devices plus swabs present therein is/are irradiated and/or autoclaved and/or gas-treated and/or sterilized so that there is a reduction in pathogens.

17. The use of a device according to the method of claim 15, wherein the packed device including the swab present therein is introduced into a cleanroom zone or into an insulator classified according to the guidelines for cleanroom classes as A, B, C or D or ISO, 8, 7, 6 or 5 or better, wherein the swab is removed from the innermost pack and critical zones or zones to be tested are sampled or swabbed using the swab, wherein the swab is then plugged back into the first housing part, wherein the ampoule is broken open at one end or at both ends, so that the solution present therein runs into the second housing part toward the swab head, wherein the liquid either flows past the rod of the swab or else flows through an inner channel in the rod of the swab, and wherein the solution is directly incubated, or immediately after use or afterwards the solution is completely or partially removed from the housing and is used for other applications.

18. The use of a device according to the method of claim 16, wherein the packed device including the swab present therein is introduced into a cleanroom zone or into an insulator classified according to the guidelines for cleanroom classes as A, B, C or D or ISO, 8, 7, 6 or 5 or better, wherein the swab is removed from the innermost pack and critical zones or zones to be tested are sampled or swabbed using the swab, wherein the swab is then plugged back into the first housing part, wherein the ampoule is broken open at one end or at both ends, so that the solution present therein runs into the second housing part toward the swab head, wherein the liquid either flows past the rod of the swab or else flows through an inner channel in the rod of the swab and wherein the solution is directly incubated, or immediately after use or afterwards the solution is completely or partially removed from the housing and is used for other applications.

19. The device according to claim 11, wherein the liquid nutrient medium for cultivation of microorganisms includes pathogens, bacteria, fungi and yeasts.

20. The device according to claim 14, wherein the medium mixed with the deinhibitor or neutralizer provided neutralization of a disinfectant or the like.
